(19) United States
(12) Patent Application Publication
(10) Pub. No.: US 2024/0299372 A1
(43) Pub. Date: Sep. 12, 2024

(54) Transdermal Drug Delivery Systems for Administration of a Therapeutically Effective Amount of Apixaban and Other Direct Oral Anticoagulants

(71) Applicant: Pike Therapeutics Inc., Vancouver (CA)

(72) Inventors: Fotios M. PLAKOGIANNIS, Whitestone, NY (US); Nisarg MODI; Rodoula PLAKOGIANNIS; Tamanna LATHER, Jersey City, NJ (US)

(21) Appl. No.: 18/594,497

(22) Filed: Mar. 4, 2024

Related U.S. Application Data
(60) Provisional application No. 63/450,515, filed on Mar. 7, 2023.

(51) Int. Cl. A61K 31/444; A61K 9/00; A61K 9/70

(52) U.S. Cl. CPC A61K 31/444; A61K 9/0021; A61K 9/7046

(57) ABSTRACT

Continuous drug delivery systems for apixaban and other direct oral anticoagulants (DOACs).

### CROSS REFERENCE

This application claims priority to U.S. Provisional Application No. 63/450,515 filed Mar. 7, 2023, titled “Transdermal Drug Delivery Systems for Administration of a Therapeutically Effective Amount of Apixaban and Other Direct Oral Anticoagulants”, the entirety of which is incorporated herein by reference.

### TECHNICAL FIELD

The present disclosure is directed to continuous drug delivery systems for apixaban and other direct oral anticoagulants (DOACs). More particularly, various embodiments are directed to provide continuous, sustained delivery of DOACs to mitigate the peak and valley pharmacokinetic behavior associated with standard immediate-release oral delivery forms, thereby improving drug efficacy and minimizing toxicity.

### BACKGROUND OF THE INVENTION

DOACs include direct thrombin inhibitor, dabigatran, and direct inhibitors of activated factor X (FXa), apixaban, rivaroxaban, edoxaban, betrixaban, representing a new generation of drugs that have been increasingly used in the prevention and treatment of thromboembolic conditions.

Apixaban (1-(4-methoxyphenyl)-7-oxo-6-[4-(2-oxopiperidin-1-yl)phenyl]-4,5-dihydropyrazolo[3,4-c]pyridine-3-carboxamide) is an FDA approved drug which is available in the form of an oral tablet. Apixaban is indicated for the prevention and treatment of thromboembolic conditions. Apixaban is available in an oral dosing form in strengths of 2.5 mg and 5 mg. Apixaban is administered twice a day.

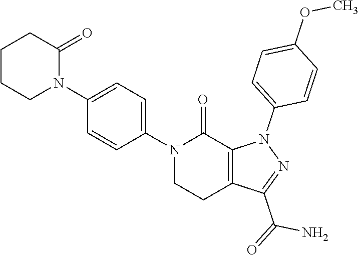 [SMILES: COc1ccc(-n2nc(C(N)=O)c3c2C(=O)N(c2ccc(N4CCCCC4=O)cc2)CC3)cc1]

Rivaroxaban (5-chloro-N-[[(5S)-2-oxo-3-[4-(3-oxomorpholin-4-yl)phenyl]-1,3-oxazolidin-5-yl]methyl]thiophene-2-carboxamide) also an FDA approved drug, which is available in the form of oral tablets. Rivaroxaban is indicated for the prevention and treatment of thromboembolic conditions. Rivaroxaban is available orally at strengths of 2.5 mg, 10 mg, 15 mg, and 20 mg.

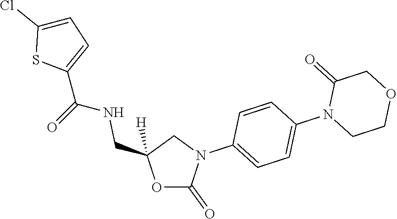 [SMILES: O=C(NC[C@H]1CN(c2ccc(N3CCOCC3=O)cc2)C(=O)O1)c1ccc(Cl)s1]

Edoxaban (N′-(5-chloropyridin-2-yl)-N-[(1S,2R, 4S)-4-(dimethylcarbamoyl)-2-[(5-methyl-6,7-dihydro-4H-[1,3]thiazolo[5,4-c]pyridine-2-carbonyl)amino]cyclohexyl]oxamide) is an FDA approved drug, which is available in the form of oral tablets. Edoxaban is indicated for the prevention and treatment of thromboembolic conditions. Edoxaban is available orally at strengths of 15 mg, 30 mg, and 60 mg.

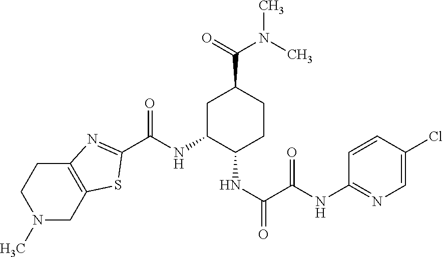 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

Betrixaban (N-(5-chloropyridin-2-yl)-2-[[4-(N,N-dimethylcarbamimidoyl) benzoyl]amino]-5-methoxybenzamide is an FDA approved drug, which is available in the form of oral tablets. Betrixaban is indicated for prophylaxis of venous thromboembolism (VTE) in adult patients hospitalized for an acute medical illness who are at risk for thromboembolic complications due to moderate or severe restricted mobility and other risk factors for VTE. Betrixaban is available in an oral dosage form at strengths of 40 mg and 80 mg.

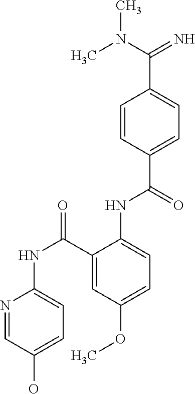 [SMILES: COc1ccc(NC(=O)c2ccc(C(=N)N(C)C)cc2)c(C(=O)Nc2ccc(O)cn2)c1]

Dabigatran, 3-[[2-[(4-carbamimidoylanilino)methyl]-1-methylbenzimidazole-5-carbonyl]-pyridin-2-ylamino]propanoic acid, is an FDA approved drug, which is available in the form of oral capsules. Dabigatran is indicated for the prevention and treatment of thromboembolic conditions. Dabigatran is available orally at strengths of 75 mg, 110 mg, and 150 mg.

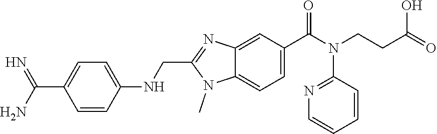 [SMILES: Cn1c(CNc2ccc(C(=N)N)cc2)nc2cc(C(=O)N(CCC(=O)O)c3ccccn3)ccc21]

Oral administration results in a cycle of high and low drug levels caused by oral administration, which are associated with unpleasant and potentially fatal side effects. For example, bleeding complications are common reasons for DOAC discontinuation (3.4-9.7% for dabigatran [1-4], 5.7-10.1% for apixaban [3-4] and 16.8-24.5% for rivaroxaban [3-4]).

DOACs are the molecules approved for the prevention and treatment of many thromboembolic conditions by targeting either thrombin or activating factor Xa. More precisely, dabigatran is the only DOAC, which is targeting thrombin, while other DOACs, such as rivaroxaban, edoxaban, and apixaban is, targeting factor Xa. DOACs have a black box warning for increased risk of stroke with discontinuation. Delivering DOACs by continuous delivery (ex., transdermal, intramuscular, subcutaneous) reduces the risk of missed doses, which may result in poor treatment outcomes, including stroke and mortality. Continuous delivery of DOACs eliminates peak blood levels associated with side effects, potentially lowering the risk of bleeding, including nosebleeds, bleeding gums, easy bruising, and bleeding taking longer to stop. The most concerning side effect and potentially fatal include internal bleeding, which may be gastrointestinal or intracranial bleeding. Further, because continuous delivery (ex., transdermal, intramuscular, subcutaneous) bypasses the gastrointestinal system, the risk of gastrointestinal bleeding may also be reduced or eliminated. The digestive system is full of intra and submucosal vascularization. The research showed that even healthy volunteer has 5% to 15% erosion in their gastric and small intestine. The root cause of gastrointestinal (GI) track damage is the presence of acid and digestive enzymes, such as amylase, trypsin, and pepsin, as well as exogenous bacterial flora, which makes intestinal vascularization prone to clinical and sub-clinical bleeding. Various mechanisms have been reported for DOACs by which they modify the bleeding conditions. 1) topical anticoagulant effect, 2) systemic anticoagulant effect, 3) caustic action at the local level, 3) topical biological effect not correlated to coagulation (inhibition of mucosal bleeding), or 4) a combination of these. The GI tract mucosal vulnerability is highly affected by the bioavailability of the drug molecules. Such as rivaroxaban is 60-80%, and apixaban is 50% bioavailable as compared to dabigatran which has only 6% bioavailability. The unabsorbed drugs pass through the intestine and are excreted into feces. During their travel through the intestinal, two-thirds of the drug is converted into an active drug by intestinal esterases. Which could have local as well as systemic effects on pre-existing mucosal vulnerability.

DOACs are the substrates of permeability glycoproteins (P-gp), a key efflux pump present in the GI tract. The transporters such as ATP binding cassettes (ABC) subfamily member 1 (ABCB1) or MDR1 (multi-drug resistance protein) are the key player in providing the protective mechanism against xenobiotics (unabsorbed drug molecules). They are widely present in the intestinal epithelium, where they pump such substrates (e.g. drugs and toxins) back into the lumen. When DOACs are administered together with a P-gp inhibitor, the decrease in the luminal efflux of the drug causes higher blood concentration of DOACs and thus increases the bleeding risk.

The research was performed in APC^{min/+} to evaluate the GI bleeding, and the model showed a significant increase in GI bleeding in the mice model treated with all the marketed DOACs through the oral route as compared to non-treatment groups.

The only way to avoid all the problems associated with GI bleeding is by incorporating the DOACs into the formulation where they can bypass the first pass metabolism or by avoiding the exposure to GI tract. The current patent application will solve the problem of GI bleeding of all the DOACs by delivering them through the transdermal and/or intramuscular and/or subcutaneous and/or mucosal and/or route which can avoid the GI exposure of the DOACs.

### Transdermal Drug Delivery

In Transdermal drug delivery, a transdermal patch or transdermal composition is applied topically to the skin surface. Sometimes upon the topical application of a transdermal patch or transdermal composition, the drug starts continuously releasing and diffusing through the intact skin (via transcellular, intercellular, and transappendageal routes) to achieve the systemic effect. Therefore, once applied, transdermal composition or transdermal patch can deliver the drug into the systemic circulation throughout the day or even for more than one day, depending on the duration of its application, which can be up to a week or longer.

Transdermal delivery can reduce the dosing frequency of DOACs (selected from the group consisting of Apixaban, Rivaroxaban, Edoxaban, Betrixaban, and Dabigatran), which is currently administered orally once a day or multiple times a day. Through transdermal delivery, transdermal compositions or transdermal patch can be applied topically to the skin, thereby delivering the drug throughout the duration of the topical application. Depending on the requirement, the topical application of transdermal patch duration can be for a few hours, for 1 day, for 2 days, for 3 days, for 4 days, for 5 days, 6 days, 7 days, or for up to 2 weeks. Therefore, transdermal delivery can overcome the multiple-dose regimen of oral delivery by reducing the dosing frequency.

Moreover, in transdermal drug delivery, the drug is delivered slowly and continuously throughout the duration of topical application, eliminating peaks and troughs in drug plasma concentration which are associated with multiple-dose administration. Therefore, transdermal delivery of DOACs can have the drug's therapeutic effect for an extended period without drastic changes in drug plasma concentration.

In the case of Apixaban, a 10 mg oral dose has an absolute bioavailability of 50%, which means not the entire orally administered dose contributes to drug efficacy. On the other hand, in transdermal delivery, the amount of drug which enters through the skin in the systemic circulation contributes towards drug efficacy. In other words, transdermally less amount of apixaban may give the same efficacy as a higher amount of orally administered apixaban. Administering apixaban transdermally allows for lower doses exposed to the body, and a longer duration of action, without peaks and troughs, compared to oral administration. Further transdermal delivery is easy, noninvasive, and convenient. Administration of transdermal patches or transdermal composition does not require medical supervision, as patients can topically apply the transdermal patch or transdermal composition themselves. Moreover, in case of any adverse effect or emergency, transdermal delivery gives the liberty to terminate the therapy at any time by removing the transdermal patch or transdermal composition from the skin.

The present disclosure provides compositions and methods that have the unique benefit of providing treatment of a condition as set forth herein that will allow a patient or heath care provider to titrate dose to the most effective dose per patient with the use, such as the addition, of an incremental dose patch.

The present disclosure provides compositions and methods which have the unique ability to immediately stop drug administration by simply removing the patch. For example, a patient may start with a dose that has the onset of unwanted negative side effects. The patient or heath care provider can control or stop administration (e.g., remove the patch, or titrate the administration with a lower dose patch) before it gets worse, and also dramatically shorten the time of the unwanted experience.

### Intramuscular Drug Delivery

In intramuscular delivery, a drug is injected into a muscle, and from the muscle, the drug releases into the systemic circulation. One type of intramuscular injection is a depot injection. Depot injections provide sustained and continuous drug delivery at a predetermined rate for a predetermined duration. Once injected, intramuscular depot injections can provide continuous delivery of the drug for a few hours, for a day, for a few days, for weeks, for a month, for more than a month. Thereby intramuscular depot injections can reduce dosing frequency. Moreover, in extended-release intramuscular drug delivery, upon injecting the drug, the drug is delivered slowly and continuously from the muscle eliminating peaks and troughs in drug plasma concentration which are associated with multiple-dose administration. Intramuscular delivery can reduce the dosing frequency of DOACs (selected from the group consisting of Apixaban, Rivaroxaban, Edoxaban, Betrixaban, and Dabigatran), which are currently administered orally once a day or multiple times a day. Through intramuscular delivery, intramuscular formulation of selected DOAC can be injected into a muscle delivering the drug continuously and slowly at a predetermined rate for a predetermined duration. Depending on the requirement, once injected, the extended-release intramuscular injection can provide continuous drug plasma concentration for a period of time selected from the group consisting of—a few hours, a day, days, weeks, month, or months. Therefore, continuous intramuscular delivery can overcome the multiple-dose regimen of oral delivery by reducing the dosing frequency.

### Subcutaneous Drug Delivery

In subcutaneous delivery, the drug is administered beneath the skin via injection or infusion. In subcutaneous injection, the drug is injected as a bolus. Subcutaneous infusion devices are used for subcutaneous drug delivery—these devices provide continuous drug supply at a predetermined rate and duration. Subcutaneous infusion devices include pumps that can be portable and wearable. Subcutaneous infusion of DOACs (selected from the group consisting of Apixaban, Rivaroxaban, Edoxaban, Betrixaban, and Dabigatran) can provide slow, continuous, and sustained delivery of selected DOAC at a predetermined rate and predetermined duration eliminating peaks and troughs in drug plasma concentration which are associated with multiple-dose administration. The duration of continuous delivery of DOAC via subcutaneous infusion can be for up to a day or multiple days, for example, for 1 day, for 2 days, for 3 days, for 4 days, for 5 days, 6 days, 7 days, or for up to 2 weeks. Compared to oral administration, administering DOAC (preferably but not limited to apixaban) transdermally, intramuscularly, and subcutaneously allows for continuous drug delivery, potentially lower drug doses exposed to the body, without or reduced plasma concentration—peaks and troughs, reduced or eliminated adverse side effects associated with peak plasma concentration, eliminating first-pass metabolism. Additionally, continuous delivery of DOAC can overcome the multiple-dose regimen of oral delivery by reducing the dosing frequency.

### Detailed Summary of Invention

As used herein, DOAC refers to a drug selected from the group consisting of Apixaban, Rivaroxaban, Edoxaban, Betrixaban, and Dabigatran either alone or in combinations thereof.

In embodiments of the disclosure, without any limitation, a preferable form of DOAC is selected from groups such as crystalline, co-crystals, polymorph, combination of polymorphs, amorphous form, the base form, pharmaceutically acceptable salts, isomers, racemic form, hydrates, anhydrous form, prodrugs, coated form, solid solution may be prepared with polymer, analogs, derivatives, active metabolites, solution form, solvates either alone or in combinations thereof.

As used herein the term “combination administration” of a compound, therapeutic agent or known drug with the combination of the present invention means administration of the drug and the one or more compounds at such time that both the known drug and/or combination will have a therapeutic effect. In some cases, this therapeutic effect will be synergistic. Such concomitant administration can involve concurrent (i.e., at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

The amorphous form of the drug does not have a definite structure. The amorphous form of the drug has a higher solubility than crystalline forms. Different techniques and methods are used to make the amorphous form of drugs.

As stated in the regulatory classification of pharmaceutical Co-Crystals guidance for Industry, “Co-crystals are crystalline materials composed of two or more different molecules, typically drug and co-crystal formers (“conformers”), in the same crystal lattice”-. Different methods are available for the preparation of Cocrystals. Each drug has a distinct chemical structure and physicochemical properties; therefore, it is difficult to predict the success rate of a cocrystallization reaction. Many experiments are required with different experimental conditions to see whether cocrystals of the drug can be prepared-

Coating of the drug can be done with polymer or other excipients. Different techniques are used for the coating of the drug. The stability of the drug can also be increased by encapsulation.

As used herein, the term “pharmaceutically acceptable salts” includes acid addition salts or addition salts of free bases. The term “pharmaceutically acceptable salts” of the DOAC within its scope all the possible isomers and their mixtures, and any pharmaceutically acceptable metabolite, bioprecursor, and/or pro-drug, such as, for example, a compound that has a structural formula different from the one of the compounds of the disclosure, and yet is directly or indirectly converted in vivo into a compound of the disclosure, upon administration to a subject, such as a mammal, particularly a human being.

Furthermore, to all their pharmaceutically acceptable forms of DOAC either alone or in combinations thereof, for example, following forms but not limited to such as free base, salts, racemic form, isomers, amorphous, crystalline, crystalline powder, polymorphs, cocrystals, solid solution, prodrugs, analogs, derivatives, metabolites, solutions, hydrates, anhydrous, solvates Therapeutic agents may be pharmaceutically acceptable salt, such as an acid addition salt, a base salt, or a solvate thereof, including a hydrate. Suitable acid addition salts are formed from acids that form nontoxic salts and without any limitation examples are acetate, hydrochloride, hydrobromide, hydroiodide, sulphate, bisulfate, nitrate, phosphate, hydrogen phosphate, sodium phosphate, maleate, fumarate, lactate, tartrate, citrate, gluconate, succinate, saccharate, benzoate, methanesulphonate, ethanesulphonate, benzenesulphonate, p-toluenesulphonate, and pamoate salts. Suitable base salts are formed from bases that form non-toxic salts and without any limitation; examples are sodium, potassium, aluminum, calcium, magnesium, zinc, and diethanolamine salts.

As used herein, the terms “subject” and “patient” are used interchangeably. As used herein, the term “patient” refers to an animal, preferably a mammal such as a non-primate (e.g., cows, pigs, horses, cats, dogs, rats, etc.) and a primate (e.g., monkey and human), and most preferably a human. In some embodiments, the subject is a non-human animal, such as a farm animal (e.g., a horse, pig, or cow) or a pet (e.g., a dog or cat). In a specific embodiment, the subject is a human. As used herein, the term “agent” refers to any molecule, compound, methodology, and/or substance used in the prevention, treatment, management, and/or diagnosis of a disease or condition. As used herein, the term “effective amount” refers to the amount of sufficient therapy to prevent the development, recurrence, or onset of a disease or condition. One or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and enhance or improve the therapeutic effect(s) of another therapy.

As used herein, the phrase “pharmaceutically acceptable” means approved by a federal or state government regulatory agency or listed in the U.S. Pharmacopeia, European Pharmacopeia, or another generally recognized pharmacopeia for use in animals, and more particularly, in humans.

As used herein, the term “therapeutic agent” refers to any molecule, compound, and/or substance used for treating and/or managing a disease or disorder.

As used herein, the terms “therapies” and “therapy” can refer to any method(s), composition(s), and/or agent(s) that can be used in the prevention, treatment, and/or management of a disease or condition, or one or more symptoms thereof. In certain embodiments, “therapy” and “therapies” refer to small molecule therapy.

The term “derivative” or “derivatized” herein includes chemical modification of a compound of the disclosure or pharmaceutically acceptable salts thereof or mixtures thereof. That is, a “derivative” may be a functional equivalent of a compound of the disclosure, capable of inducing improved pharmacological functional activity in a given subject.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “about” when used in conjunction with a stated numerical value or range has the meaning reasonably ascribed to it by a person skilled in the art, i.e., denoting somewhat more or somewhat less than the stated value or range.

As used herein, continuous delivery refers to a drug delivery system selected from a group consisting of transdermal drug delivery, subcutaneous delivery, and extended-release intramuscular delivery. As used herein, continuous delivery refers to the uninterrupted administration of DOAC to the subject via transdermal delivery or extended intramuscular delivery or subcutaneous delivery, such as but not limited to subcutaneous infusion or subcutaneous depot extended-release intramuscular delivery wherein the duration of DOAC continuous delivery is predetermined. In some embodiments, the continuous delivery system of DOAC described herein provides a constant delivery rate of the DOAC over a predetermined period. In some embodiments, the predetermined period is 0-4 hr, 4-24 hr, 24 hours, 48 hours, 72 hours, 96 hours, 120 hours, 144 hours, 7 days, 8 to 13 days, two weeks, or 15 days, 15 days-30 days, more than 30 days.

In yet further embodiments, the continuous delivery system of DOAC described herein provides a steady DOAC plasma concentration to subject over a predetermined time. In some embodiments, the predetermined period is selected from anytime such as but limited to 0-24 hrs, 24 hours, 48 hours, 72 hours, 96 hours, 120 hours, 144 hours, 7 days, 8 to 13 days, two weeks, or 15 days, 15 days-30 days, more than 30 days.

In yet further embodiments, the continuous delivery system of DOAC described herein provides a sustained DOAC plasma concentration to subject over a predetermined time. In some embodiments, the predetermined period is selected from anytime such as but limited to 0-24 hrs, 24 hours, 48 hours, 72 hours, 96 hours, 120 hours, 144 hours, 7 days, 8 to 13 days, two weeks, or 15 days, 15 days-30 days, more than 30 days.

In yet further embodiments, the continuous delivery system of DOAC described herein provides a DOAC plasma concentration to subject over a predetermined time. In some embodiments, the predetermined period is selected from anytime such as but limited to 0-24 hrs, 24 hours, 48 hours, 72 hours, 96 hours, 120 hours, 144 hours, 7 days, 8 to 13 days, two weeks, or 15 days, 15 days-30 days, more than 30 days.

In yet further embodiments, the continuous delivery system of DOAC described herein provides a continuous DOAC plasma concentration to subject over a predetermined time. In some embodiments, the predetermined period is selected from anytime such as but limited to 0-24 hrs, 24 hours, 48 hours, 72 hours, 96 hours, 120 hours, 144 hours, 7 days, 8 to 13 days, two weeks, or 15 days, 15 days-30 days, more than 30 days yet further embodiments, the continuous delivery systems of DOAC described herein provide a DOAC plasma concentration in a therapeutic range to subject over a predetermined time. In some embodiments, the predetermined period is selected from anytime such as but limited to 0-24 hrs, 24 hours, 48 hours, 72 hours, 96 hours, 120 hours, 144 hours, 7 days, 8 to 13 days, two weeks, or 15 days, 15 days-30 days, more than 30 days

In yet further embodiments, the continuous delivery system of DOAC described herein provide a therapeutically effective plasma concentration of DOAC lower than oral peak plasma concentration thereby continuous delivery may eliminate or reduceDOAC adverse effects associated with oral peak plasma concentration. Furthermore, the continuous delivery systems of DOAC described herein provide a therapeutically effective plasma concentration of DOAC lower than oral peak plasma concentration to subject over a predetermined time. In some embodiments, the predetermined period is selected from anytime such as but limited to 0-24 hrs, 24 hours, 48 hours, 72 hours, 96 hours, 120 hours, 144 hours, 7 days, 8 to 13 days, two weeks, or 15 days, 15 days-30 days, more than 30 days.

In yet further embodiments, the continuous delivery systems of DOAC described herein allow for reduced variability in the dosage of DOAC in a subject over a predetermined time. In some embodiments, the predetermined time is 24 hours, 48 hours, 72 hours, 96 hours, 120 hours, 144 hours, 7 days, 8 to 13 days, two weeks, or 15 days, 15 days-30 days, more than 30 days.

In yet further embodiments, the continuous delivery systems of DOAC described herein can have lag time in DOAC plasma concentration in subjects wherein lag time is the time between drug administration and seeing plasma concentration in subject. In some embodiments, the lag time is anytime between 0-24 hours, 0-48 hours, 0-72 hours, 0-120 hours.

In yet further embodiments, the continuous delivery systems of DOAC described herein which may provide steady DOAC plasma concentration, sustained DOAC plasma concentration, continuous DOAC plasma concentration, DOAC plasma concentration in therapeutic range may have lag time in DOAC plasma concentration in subjects wherein lag time is the time between drug administration and seeing plasma concentration in subject. In some embodiments, the lag time is anytime between 0-24 hours, 0-48 hours, 0-72 hours, 0-120 hours.

In yet further embodiments, the continuous delivery system of DOAC described herein provide a slow and sustained plasma concentration of DOAC avoiding peaks and troughs in plasma concentration associated with oral delivery.

In one aspect the continuous delivery systems of DOAC can have a dosage regimen selected from the group such as but not limited to administer once a day, once in two days, once in three days, once in four days, once in five days, once in six days, once in a week, once in 7-10 days, once in 10-15 days, once in about a 15-30 days, once in about 30 days-90 days.

In oral administration drug is absorbed in the gastrointestinal tract. Absolute oral bioavailability of apixaban is approx. 50%, rivaroxaban is approx. 80%-100%, edoxaban is approx. 62%, betrixaban is approx. 34%, dabigatran is approx. 3%-7%. After oral administration DOAC such as apixaban, edoxaban, betrixabam, dabigatran have less than 100% bioavailability which means not the entire orally administered dose is reaching systemic circulation and contributing the drug efficacy. Therefore, body is exposed to more oral dose than the dose which is bringing the efficacy. In continuous delivery system the amount of drug enters the systemic circulation via skin in transdermal delivery, via subcutaneous tissue in subcutaneous delivery and from muscle in intramuscular delivery. In continuous delivery system drug reaches systemic circulation bypassing first pass metabolism. In continuous delivery less amount of DOAC may give the same efficacy as higher amount of orally administered DOAC. Continuous administration of DOAC allows for lower doses exposed to the body.

As used herein, the terms “composition” and “formulation” and “delivery systems” are used interchangeably

As used herein, the term “topical delivery” means the delivery of the drug into systemic circulation through the skin.

In one embodiment continuous delivery of DOAC is selected from group of continuous delivery systems or formulations consisting of transdermal delivery, subcutaneous infusion, extended intramuscular delivery, wherein DOAC is selected from the group consisting of Apixaban, Rivaroxaban, Edoxaban, Betrixaban, Dabigatran either alone or in combinations thereof.

In one aspect formulation for continuous delivery of DOAC is selected from group consisting of solid formulations, semisolid formulations, liquid formulations, spray formulation.

In another aspect formulation for transdermal delivery of DOAC is selected from the group consisting of transdermal delivery systems such as but not limited to transdermal matrix patch, transdermal reservoir patch, topical formulations wherein transdermal matrix patch is selected from the group consisting of but not limited to a transdermal adhesive matrix patch, transdermal adhesive matrix patch with peripheral adhesives or overlay adhesives, transdermal polymer patch, transdermal polymer patch with peripheral adhesive or overlay adhesives, pressure sensitive adhesive matrix patch with or without peripheral adhesive or overlay adhesive, single layer or multiple layers matrix patch with or without peripheral or overlay adhesives, single or multiple layer pressure sensitive adhesive matrix patch with or without peripheral or overlay adhesive, monolithic patches with or without adhesives or peripheral adhesives or overlay adhesives, hydrogel matrix patches, drug in adhesive patches, microreservoir patches. Furthermore, formulation for the transdermal matrix patch is selected from the group consisting of but not limited to solution, suspensions, homogenous mixture, dispersions, emulsions, microemulsions, hydrogels etc. The formulation for transdermal reservoir patch is selected from the group but not limited to semisolid formulations, liquid formulations such as but not limited to gels, emulsions, microemulsions, nanosuspension, nanoparticle formulations, solutions, suspensions, nanoemulsion, dispersions, micelles, polymer solutions, etc. Reservoir patches can have peripheral adhesives or overlay adhesive. Topical formulations for transdermal delivery of DOAC is selected from the group consisting of but not limited to paste, solutions, suspensions, dispersions, emulsions, creams, gels, polymer solutions, ointments, balms, sprays, metered dose spray, metered dose pumps, film forming semisolid formulations, film forming liquid formulations, aerosols, etc. Other formulations for transdermal delivery of DOAC can be selected from the group, such as but not limited to microneedles, microblades, extended-release films, sustained-release films, iontophoresis topical tapes comprising formulations, and other well-known transdermal delivery systems or transdermal formulations well known to those skilled in the art. Without any limitation, transdermal delivery system can be occlusive, semi-occlusive, or nonocclusive and can be adhesive or non-adhesive.

In yet another aspect formulations for continuous intramuscular delivery, continuous subcutaneous delivery of DOAC is selected from the group known to those skilled in the art such as but not limited to depots, in situ forming implants, in situ biodegradable implant, in situ gel-forming implants, lipid-based formulation, solutions, suspensions, microspheres, biodegradable microspheres, lipid-based formulations, liposomes, liquid formulations, polymeric nano/microparticles, subcutaneous pellet implants, subcutaneous infusion pumps, etc., subcutaneous implants, subcutaneous infusion devices, etc. Formulation for continuous subcutaneous delivery will be delivered via such as but not limited to subcutaneous infusion devices, subcutaneous infusion pumps, etc. Other formulations for continuous intramuscular delivery and continuous subcutaneous delivery known to those skilled in the art are also within the scope of this provisional patent application.

In certain embodiments the active agent is provided at a concentration equal to or greater than about 90%, 91%, 92%, 93%, 94%, 95%, 96%, 97%, 98%, 99%, 99.5%, 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the active agent is for example, a DOAC, or for example, a combination of DOACs, or for example DOACs and other medications. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, or 45 mg/kg/day. In certain embodiments, the active agent is titrated from 5 to 20-25 mg/kg/day and optionally maintained for 10-15 days. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg. In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% of the formulation. In exemplary embodiments, formulations of the disclosure may comprise active agent at a concentration of about 0.1 to about 50%, about 1 to 20%, of about 5% to 25%, about 10% to about 20%, or about 15% to about 18%, about 30% to about 70%, about 35% to about 65%, about 63.13%, about 40% to about 64% w/w, about 95 to about 98%, or about 95 to about 97%. In exemplary formulations of the disclosure, the active agent will represent approximately about 1% to about 10%, about 1% to about 9%, about 1% to about 8%, about 1% to about 7%, about 1% to about 6%, about 1% to about 5%, about 0.1 wt % to about 50% wt %, 1 wt % to 75 wt %, preferably 2 wt % to 30 wt %, more preferably 5 wt. % to 20 wt. % of the formulation.

In yet further embodiments, the pharmaceutical compositions described herein provide a plasma concentration of the active agent components in a therapeutic range in a patient over a predetermined time. In exemplary embodiments as disclosed herein, the pharmaceutical composition provides a blood serum level of active agent selected from without any limitation, of, for example, about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, about 5 μg/mL, and ranges thereof. In one aspect, the pharmaceutical composition provides a blood serum level of active agent in the range of 0.01 ng/mL-400 ng/mL. In another aspect, the formulations as disclosed herein provides a blood serum level of active agent in the range of 0.01 ng/mL-100 ng/mL. In yet another aspect the pharmaceutical composition provides a blood serum level of active agent in the range of from 0.01-1 ng/ml to 1-100 ng/ml to 100-500 ng/ml to 500-1000 ng/ml to 1000-5000 ng/ml.

In yet further embodiments, the pharmaceutical compositions described herein provide a plasma concentration of the active agent components in a therapeutic range in a patient over a predetermined time. In exemplary embodiments as disclosed herein, the pharmaceutical composition provides a blood serum level of active agent selected from without any limitation, of, for example, about 0.01 microgm/mL, about 0.02 microgm/mL, about 0.05 microgm/mL, about 0.1 microgm/mL, about 0.2 microgm/mL, about 0.5 microgm/mL, about 1 microgm/mL, about 2 microgm/mL, about 5 microgm/mL, about 10 microgm/mL, about 20 microgm/mL, about 50 microgm/mL, about 100 microgm/mL, about 200 microgm/mL, about 500 microgm/mL, about 1 milligm/mL, about 2 milligm/mL, about 5 milligm/mL, and ranges thereof. In one aspect, the pharmaceutical composition provides a blood serum level of active agent in the range of 0.01 microgm/mL-400 microgm/mL. In another aspect, the pharmaceutical composition provides a blood serum level of active agent in the range of 0.01 microgm/mL-100 microgm/mL. In yet another aspect the pharmaceutical composition provides a blood serum level of active agent in the range of from 0.01-1 microgm/mL to 1-100 microgm/mL to 100-500 microgm/mL to 500-1000 microgm/mL to 1000-5000 microgm/mL.

In one embodiment described herein, the continuous delivery formulation of DOAC contains Apixaban in the form selected from groups such as but not limited to cocrystals, amorphous form, crystalline form, crystalline powder, coated form, its solution, its solid solution, its polymorphs, combination of polymorphs and its salts which can be anhydrous and/or hydrous, racemic forms, isomers, solvates, derivatives alone or in combinations thereof wherein continuous delivery of apixaban will be selected from the group consisting of transdermal delivery, continuous subcutaneous delivery, continuous intramuscular delivery. The desired optimum continuous delivery formulation of apixaban may comprise without any limitation to following carriers as stated from example 1 to example 11 either alone or in combinations thereof. Apixaban may be dissolved, suspended, dispersed, or uniformly mixed in the continuous delivery formulation. Concentration of apixaban in the continuous delivery formulation is selected from the range of 0.01%-99.99% w/w or w/v, for example about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% w/w of the formulation.

In another embodiments described herein the continuous delivery formulation of DOAC contains Rivaroxaban in the form selected from groups such as but not limited to cocrystals, amorphous form, crystalline form, crystalline powder, coated form, its solution, its solid solution, its polymorphs, combination of polymorphs and its salts which can be anhydrous and/or hydrous, racemic forms, isomers, solvates, derivatives alone or in combinations thereof wherein continuous delivery of rivaroxaban will be selected from the group consisting of transdermal delivery, continuous subcutaneous delivery, continuous intramuscular delivery. The desired optimum continuous delivery formulation of rivaroxaban may comprise without any limitation to following carriers as stated from example 1 to example 11 either alone or in combinations thereof. rivaroxaban may be dissolved, suspended, dispersed, or uniformly mixed in the continuous delivery formulation. Concentration of rivaroxaban in the continuous delivery formulation is selected from the range of 0.01%-99.99% w/w or w/v, for example about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% w/w of the formulation.

In yet another embodiments described herein the continuous delivery formulation of DOAC contains edoxaban in the form selected from groups such as but not limited to cocrystals, amorphous form, crystalline form, crystalline powder, coated form, its solution, its solid solution, its polymorphs, combination of polymorphs and its salts which can be anhydrous and/or hydrous, racemic forms, isomers, solvates, derivatives alone or in combinations thereof wherein continuous delivery of edoxaban will be selected from the group consisting of transdermal delivery, continuous subcutaneous delivery, continuous intramuscular delivery. The desired optimum continuous delivery formulation of edoxaban may comprise without any limitation to following carriers as stated from example 1 to example 11 either alone or in combinations thereof. Edoxaban may be dissolved, suspended, dispersed, or uniformly mixed in the continuous delivery formulation. Concentration of edoxaban in the continuous delivery formulation is selected from the range of 0.01%-99.99% w/w or w/v, for example about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% w/w of the formulation.

In yet another embodiment described herein the continuous delivery formulation of DOAC contains Betrixaban in the form selected from groups such as but not limited to cocrystals, amorphous form, crystalline form, crystalline powder, coated form, its solution, its solid solution, its polymorphs, combination of polymorphs and its salts which can be anhydrous and/or hydrous, racemic forms, isomers, solvates, derivatives alone or in combinations thereof wherein continuous delivery of Betrixaban will be selected from the group consisting of transdermal delivery, continuous subcutaneous delivery, continuous intramuscular delivery.

The desired optimum continuous delivery formulation of Betrixaban may comprise without any limitation to following carriers as stated from example 1 to example 11 either alone or in combinations thereof. Betrixaban may be dissolved, suspended, dispersed, or uniformly mixed in the continuous delivery formulation. Concentration of betrixaban in the continuous delivery formulation is selected from the range of 0.01%-99.99% w/w or w/v, for example about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% w/w of the formulation.

In yet another embodiments described herein the continuous delivery formulation of DOAC contains Dabigatran in the form selected from groups such as but not limited to cocrystals, amorphous form, crystalline form, crystalline powder, coated form, its solution, its solid solution, its polymorphs, combination of polymorphs and its salts which can be anhydrous and/or hydrous, racemic forms, isomers, solvates, derivatives alone or in combinations thereof wherein continuous delivery of Dabigatran will be selected from the group consisting of transdermal delivery, continuous subcutaneous delivery, continuous intramuscular delivery.

The desired optimum continuous delivery formulation of Dabigatran may comprise without any limitation to following carriers as stated from example 1 to example 11 either alone or in combinations thereof. Dabig r lower drug exposure and lowering adverse effects associated with Cmax.atarn may be dissolved, suspended, dispersed, or uniformly mixed in the continuous delivery formulation. Concentration of dabigatarn in the continuous delivery formulation is selected from the range of 0.01%-99.99% w/w or w/v, for example about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% w/w of the formulation.

In yet another embodiments described herein the continuous delivery formulation contains combination of DOAC, that is-contains more than one DOAC wherein DOAC is selected from the group consisting of apixaban, rivaroxaban, edoxaban, betrixaban, dabigatran wherein apixaban, rivaroxaban, edoxaban, betrixaban, dabigatran are selected from groups such as but not limited to cocrystals, amorphous form, crystalline form, crystalline powder, coated form, its solution, its solid solution, its polymorphs, combination of polymorphs and its salts which can be anhydrous and/or hydrous, racemic forms, isomers, solvates, derivatives alone or in combinations thereof wherein continuous delivery of combination of DOAC will be selected from the group consisting of transdermal delivery, continuous subcutaneous delivery, continuous intramuscular delivery. The desired optimum continuous delivery formulation of DOAC combination may comprise without any limitation to following carriers as stated from example 1 to example 11 either alone or in combinations thereof. DOAC combination may be dissolved, suspended, dispersed, or uniformly mixed in the continuous delivery formulation.

Concentration of DOAC combination in the continuous delivery formulation is selected from the range of 0.01%-99.99% w/w or w/v, for example about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% w/w of the formulation

The DOAC (selected from the group apixaban, rivaroxaban, edoxaban, betrixaban, dabigatran either alone or in combinations thereof) continuous delivery formulation of the present invention may include carriers or ingredients in effective amounts either alone or in combinations thereof without any limitation to the following carriers or ingredients such as solvents, oils, gelling agents, polymers, biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, pressure sensitive adhesive polymers, diluents, tonicity agents, excipients, preservatives, material to prepare a patch, material to prepare matrix patch, material to prepare reservoir patch, excipients to make subcutaneous implants, excipients to make subcutaneous infusion formulation, excipients to make continuous subcutaneous formulation, excipients to make continuous intramuscular formulation, etc.

### EXAMPLES

### Example 1

The continuous delivery formulation of the disclosure may comprise, or exclude, solvents, cosolvents known to those skilled in the art either alone or in combinations thereof without any limitation to following like alcohol C₁-C₂₀ such as but not limited to (methanol, ethanol, isopropyl alcohol, butanol, propanol etc.), deionized water, polyhydric alcohols, glycols such as but not limited to (propylene glycol, polyethylene glycol, PEG 300, PEG 400, dipropylene glycol, hexylene glycol, butyene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils such as but not limited to (castor oil, sesame oil, cottonseed oil, coconut oil, etc.), water, polar solvents, semi polar solvents, non polar solvents, volatile chemicals to prepare transdermal delivery systems such as but not limited to (ethanol, propanol, ethyl acetate, acetone, methanol, dichloromethane, chloroform, toluene, IPA, tetrahydrofuran), acids such as but not limited to acetic acid, lactic acid, levulinic acid, bases, complexing agents, cyclodextrins, 2-hydroxypropyl-beta cyclodextrin, cremophor EL and others. More preferably in the range of 0.01%-99.9% w/w or w/v. In exemplary embodiments, formulations of the disclosure may comprise solubilizers, surfactants, emulsifying agents, dispersing agents and similar compounds or chemicals at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds or chemicals at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the solubilizers, surfactants, emulsifying agents, dispersing agents and similar compounds or chemicals will represent approximately 1 wt % to 75 wt %, preferably 2 wt % to 30 wt %, more preferably 5 wt. % to 20 wt. % of the formulation.

### Example 2

The continuous delivery formulation of the disclosure may comprise, or exclude, gelling agents, thickening agent, suspending agents, viscosity enhancing agents, dispersing agents, pressure sensitive adhesive polymers, polymers, biodegrabable polymers known to those skilled in the art either alone or in combinations thereof without any limitation to following like natural polymers, polysaccharides, biodegradable polymers, gums and its derivatives such as but not limited to (agar, alginic acid and derivatives, cassia tora, collagen, gelatin, gellum gum, guar gum, locust bean gum, pectin, gum arabic, potassium, or sodium carageenan, tragacanth, xantham, gum copal, chitosan, resin, dextran, starch, polyacrylic acid, polyvinyl, pullulan, karaya gum, dextran, alginic acid, mannan, etc.), semisynthetic and synthetic polymers and its derivatives such as without any limitation to cellulose and its derivatives (methylcellulose, ethyl cellulose, carboxymethyl cellulose, hydroxylpropyl cellulose, hydroxylpropylmethyl cellulose, microcrystalline cellulose hydroxymethyl cellulose, HPMC AS, hydroxy ethyl cellulose, propylmethyl cellulose phthalate, carboxyvinyl polymers or carbomers (carbopol 940, carbopol 934, carbopol 971p NF, sodium carboxymethyl cellulose, cellulose acetate phthalate), polyethylene, and its copolymers etc, clays such as but not limited to (silicates, bentonite), silicon dioxide, polyvinyl alcohol, polyvinyl alcohol and its derivatives, acrylic polymers, ammonioalkyl methacrylate copolymers (eudragit), acrylic acid esters, polyacrylate copolymers, polyacrylamide, polyvinylpyrrolidone and its derivatives, polyvinyl pyrrolidone homopolymer and polyvinyl pyrrolidone copolymers such as but not limited to (PVP, Kollidon 30, kollidon 30LP kollidon 12, Kollidon V A 64, Kollidon 90, poloxamer), ethylene and its derivatives, propylene and its derivatives, Soluplus, isobutylene, ethyl vinyl acetate copolymers, natural rubber, synthetic rubber, bentonite, all water and/or organic solvent swellable polymers pressure sensitive adhesives such as silicone polymers such as but not limited to (BIO-PSA® 7-4602, BIO-PSA® 7-4502, BIO-PSA® 7-4601, BIO-PSA® 7-4402, BIO-PSA® 7-4501, BIO-PSA® 7-4401 (Dow Corning@, Dow Chemicals (Dupont) Midland M I), silicone pressure sensitive adhesive polymers compatible with amine group include, without limitation, one or more of BIO-PSA® 7-4302, BIO-PSA® 7-4202, BIO-PSA® 7-4301, BIO-PSA® 7-4102, BIO-PSA® 7-4201, BIO-PSA® 7-4101. etc.), acrylate copolymer pressure sensitive adhesives with or without vinyl acetate, without or with functional group wherein functional groups are carboxyl functional group, hydroxyl functional group, with or without crosslinker such as but not limited to (Duro-Tak® 87-2196, Duro-Tak® 87-2194, Duro-Tak® 87-9301, Duro-Tak® 87-4098, Duro-Tak® 87-2051/387-2051, Duro-Tak®87-2852/387-2052, Duro-Tak® 387-2054/87-2054, Duro-Tak® 87-2854, Gelva GMS 3083, Gelva GMS 788, Duro-Tak® 87-2510/387-2510, Gelva GMS 9073, Duro-Tak® 87-2852, Duro-Tak® 87-235 A, Duro-Tak® 87-2353/387-2353, Duro-Tak® 87-4287, Duro-Tak® 87-2287/387-2287, Duro-Tak® 387-2516/87-2516, Duro-Tak@ 87-9301, etc. (Henkel)., etc.), polyisobutylene/polyisobutene adhesive such as but not limited to (polyisobutylene low molecular weight, plyisobutylene medium molecular weight, polyisobutylene high molecular weight such as but not limited to polyisobutylene 2300 MW, 550000 MW, 800000 MW, 35000 MW, 1100000 MW, or mixtures thereof), Duro-Tak@ 87-6908, etc), acrylic copolymers, rubber based adhesives, hot melt adhesives, styrene-butadiene styrene, styrene isoprene styrene, etc. Biodegradable polymers such as but not limited to (polymers or copolymers composed of monomers of lactic acid and glycolic acids, polyglycolides (PGA), poly(s-caprolactone) (PCL), polylactides (PLA), poly(s-caprolactone) (PCL), poly(s-caprolactone) (PCL), polyanhydrides, poly(dioxanone), polyglyconate, polyalkylcyanoacrylates, polyorthoesters, etc.), More preferably in the range of 0.1%-99.9% w/w or w/v. In exemplary embodiments, formulations of the disclosure may comprise gelling agents and/or thickening and/or suspending agents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise gelling agents and/or thickening and/or suspending agents at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the gelling agents and/or thickening and/or suspending agents will represent approximately 1 wt % to 75 wt %, preferably 2 wt % to 30 wt %, more preferably 5 wt. % to 20 wt. % of the formulation.

### Example 3

The continuous delivery formulation of the disclosure may comprise, or exclude, permeation/penetration enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate, Lauryl Lactate, etc), fatty acids and its derivatives with carbon chain length from C4-C26 such as but not limited to (capric acid, caprylic acid, lauric acid, oleic acid, myristic acid, linoleic acid, stearic acid, palmitic acid etc.), alcohols, fatty alcohols and its derivatives and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate, Laureth, Cetheth, oleth, ceteareth, etc.), terpene, terpenoids and all penetration or permeation enhancers referred in the book “Percutaneous Penetration Enhancers” (Eric W. Smith, Howard I. Maibach, 2005. November, CRC press). More preferably in the range of 0.01%-99.9% w/w or w/v. In exemplary embodiments, formulations of the disclosure may comprise permeation enhancer(s) at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise penetration or permeation enhancer(s) at a concentration of about 1 to 20%, of about 5% to 25%, about 10% to about 20%, or about 15% to about 18%, about 30% to about 70%, about 35% to about 65%, and about 40% to about 64% w/w. In exemplary formulations ofthe disclosure, the permeation enhancer(s) will represent approximately 1 wt % to 75 wt %, preferably 2 wt % to 30 wt %, more preferably 5 wt. % to 20 wt. % of the formulation.

### Example 4

The continuous delivery formulation of the disclosure may comprise, or exclude, plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters, fatty acids, fatty alcohols, surfactants and all the plasticizers which can be used in transdermal drug delivery system referred in the book “Handbook of Plasticizers” (George Wypych, 2004, Chem Tec Publishing). More preferably in the range of 0.01%-99.9% w/w or w/v. In exemplary embodiments, formulations of the disclosure may comprise plasticizer(s) at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizer(s) at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the plasticizer(s) will represent approximately 1 wt % to 75 wt %, preferably 2 wt % to 30 wt %, more preferably 5 wt. % to 20 wt. % of the formulation.

### Example 5

The continuous delivery formulation of the disclosure may comprise, or exclude, emollients, humectants, skin irritation reducing agents and the similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-99.9% w/w or w/v. In exemplary embodiments, formulations of the disclosure may comprise emollients, humectants, skin irritation reducing agents and the similar compounds or chemicals at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and the similar compounds or chemicals at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the emollients, humectants, skin irritation reducing agents and the similar compounds or chemicals will represent approximately 1 wt % to 75 wt %, preferably 2 wt % to 30 wt %, more preferably 5 wt. % to 20 wt. % of the formulation.

### Example 6

The continuous delivery formulation of the disclosure may comprise, or exclude, solubilizers, surfactants, emulsifying agents, auxiliary emulsifying agent dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl-8 glycerides etc, cyclodextrins, Brij surfactants, oleth surfactants, oleth, kollidon CLM and others, polyethylene glycol (molecular weight 200 g/mol-35000 g/mol. Depending on molecular weight polyethylene glycol can function as surfactant and/or thickening agent). More preferably in the range of 0.01%-99.9% w/w or w/v. In exemplary embodiments, formulations of the disclosure may comprise solubilizers, surfactants, emulsifying agents, dispersing agents and similar compounds or chemicals at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds or chemicals at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the solubilizers, surfactants, emulsifying agents, dispersing agents and similar compounds or chemicals will represent approximately 1 wt % to 75 wt %, preferably 2 wt % to 30 wt %, more preferably 5 wt. % to 20 wt. % of the formulation.

### Example 7

Different techniques and ingredients can be used to increase the stability and/or solubility of DOAC continuous delivery formulation such as without any limitation to coating, encapsulation, microencapsulation, nanoencapsulation, lyophilization, chelating agents, complexing agents, preparation of oleogels, etc.

### Example 8

The continuous delivery formulation of the disclosure may comprise, or exclude, auxiliary pH buffering agents and pH stabilizers, tonicity agents and similar compounds known to those skilled in the art which helps to maintain the appropriate pH of formulation preferably in the range of 2.0-11.0 either alone or in combination thereof without any limitation to following such as buffers which can maintain pH in the range selected from 2.0-11.0 (such as but not limited to tartrate buffer, malate buffer, lactate buffer, gluconate buffer, maleate buffer, succinate buffer, phosphate buffer, acetate buffer, citrate buffer, glycine buffer, histidine buffer, TRIS, etc)., acids, weak organic acids such as but not limited to (carboxylic acids, inorganic acids, sulfonic acids, vinylogous carboxylic acids, citric acid, phosphoric acid, hydrochloric acid, acetic acid, succinic acid and others), base, weak bases such as but not limited to (sodium hydroxide, potassium hydroxide, ammonium hydroxide, triethylamine, sodium carbonate, sodium bicarbonate, tromethamine, calcium hydroxide), tonicity agents such as but not limited to sodium chloride, dextrose, mannitol, potassium chloride, etc, excipients to impart optimum osmolarity to formulations etc. More preferably in the range of 0.01%-30% w/w or w/v. In exemplary embodiments, formulations of the disclosure may comprise auxiliary pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise auxiliary pH buffering agents and pH stabilizers and similar compounds at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the auxiliary pH buffering agents and pH stabilizers and similar compounds will represent approximately 1 wt % to 75 wt %, preferably 2 wt % to 30 wt %, more preferably 5 wt. % to 20 wt. % of the formulation. In certain embodiments, the pH of the formulation is maintained at about 4.0, about 4.5, about 5.0, about 5.5, about 6.0, about 6.5, about 7.0, about 7.5, or about 8.0. In certain embodiments, the pH of the formulation is maintained at a range of about 4.0 to about 8.0, about 4.5 to about 7.5, or about 5.0 to about 7.0.

### Example 9

The continuous delivery formulation of the disclosure may comprise, or exclude, stabilizers, antioxidants, preservatives such as but not limited to (sodium metabisulfite, citric acid, ascorbic acid, BHA, BHT, ascorbyl palmitate, alpha tocopherol), oxidizing agents, stabilizers, discoloring agents, preservatives (such as but not limited to methyl paraben, propyl paraben, benzyl alcohol, phenoxyethanol, etc.) and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation.

### Example 10

The continuous delivery formulation of the disclosure may comprise, or exclude, Diluents and fillers (such as but not limited to dextrose, lactose, mannitol, starch, talc, silicone dioxide, colloidal silicon dioxide, clays, titanium dioxide, sugars, kaolin etc.), tackifiers, crystallization inhibitors, cross linking agents, clays, etc., other well-known excipients to make transdermal formulation, other well-known excipients to make intramuscular formulation for continuous delivery, other well-known excipients to make subcutaneous formulation for continuous delivery are also within the scope of this invention. The transdermal formulation of the disclosure may be formulated in ointment and/or cream base known to those skilled in the art.

### Example 11

Materials to make the transdermal delivery system of the disclosure in patch form known to those skilled in the art, for example, such as but not limited to reservoir patch, matrix patch, drug in adhesives, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid—isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, backing membrane also includes fabrics, non-woven materials etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The continuous transdermal delivery system of the disclosure may deliver at least therapeutic effective dose of DOAC. Furthermore, the precise therapeutic effective dose of DOAC in transdermal delivery system can be determined by those skilled in the art based on factors such as but not limited to the patient's condition etc. The transdermal delivery system will be available in different dosage strengths and patch sizes to achieve optimum therapeutic outcome based on patient's requirement. If needed multiple transdermal delivery systems can be applied once to skin. In certain embodiments of the present invention, a transdermal delivery system comprises a transdermal formulation containing DOAC that allows the transdermal delivery system to adhere to the skin or transdermal delivery system topically applied to skin, allowing the passage of the DOAC from the transdermal delivery system through the skin of the patient. The transdermal delivery system can be occlusive, semi-occlusive, or nonocclusive, and can be adhesive or non-adhesive.

The continuous subcutaneous delivery system of the disclosure includes without any limitation subcutaneous infusion system. DOAC subcutaneous infusion of the disclosure may deliver slow, continuous therapeutic effective dose of DOAC. Furthermore, the precise therapeutic effective dose of DOAC in subcutaneous infusion can be determined by those skilled in the art based on factors such as but not limited to the patient's condition etc. The rate and duration of subcutaneous delivery of DOAC can be determined by infusion system. There are various ambulatory subcutaneous infusion pumps well known to those skilled in the art. Subcutaneous depot injections which can provide continuous and slow delivery of DOAC are also within the scope of this invention.

Known to those skilled in the art excipient can have multifunction exemplary same excipient can act as a solvent, penetration enhancer and plasticizer. Furthermore, same excipient can act as a penetration enhancer in transdermal formulations and solubilizer or surfactant in continuous subcutaneous formulation and continuous intramuscular formulation.

Other continuous delivery system of DOAC known to those skilled in the art such as but not limited to intravenous infusion, buccal, slow-release oral dosage form, extended release oral dosage form, sustained release oral dosage form, implants, etc. Other delivery system of DOAC which can bypass GI absorption known to those skilled in the art such as but not limited to rectal, sublingual, etc. are also within the scope of this invention.

### REFERENCES

1. Jackson L R, Kim S, Shrader P et al. Early therapeutic persistence on dabigatran versus warfarin therapy in patients with atrial fibrillation: results from the outcomes registry for better informed treatment of atrial fibrillation (ORBIT-AF) registry. J Thromb Thrombolysis 2018; 46: 435-9.
2. O'Brien E C, Simon D N, Allen L A et al. Reasons for warfarin discontinuation in the outcomes registry for better informed treatment of atrial fibrillation (ORBIT-AF). Am Heart J 2014; 168: 487-94.
3. Naganuma M, Shiga T, Nagao T et al. Renal function and treatment persistence with non-vitamin K antagonist oral anticoagulants in Japanese patients with atrial fibrillation: a single-center experience. Jpn J Clin Pharmacol Ther 2016; 47: 115-22.
4. Shiga T, Naganuma M, Nagao T et al. Persistence of non-vitamin K antagonist oral anticoagulant use in Japanese patients with atrial fibrillation: a single-center observational study. J Arrhythm 2015; 31: 339-44.
5. Sustained-Release Injectable Drug Delivery (pharmtech.com), accessed on Feb. 27, 2023.
6. FORMULATION FORUM—Rational Design & Development of Long-Acting Injectable Dosage Forms (drug-dev.com), accessed on Feb. 27, 2023.
7. Sustained-Release Injectable Drug Delivery (pharmtech.com), accessed on Feb. 28, 2023.
8. CDER “Regulatory classification of pharmaceutical co-crystals guidance for industry” Feburary 2018 Phamaceutical quality/CMC Regulatory Classification of Pharmaceutical Co-Crystals Guidance for Industry (fda.gov), Accessed on Mar. 2, 2023.

1. A method of preventing and treating thromboembolic conditions by continuous administration of the treatment of formulations comprising a Direct Oral Anticoagulants (DOACs) compound and a pharmaceutically acceptable carrier, where the DOACs compound is selected from the group consisting of DOACs, and wherein the method comprises continuously administrating their formulation to the subject via transdermal and/or subcutaneous and/or intramuscular and/or buccal route and/or by avoiding the gastrointestinal (GI) tract exposure.

2. The method of claim 1, wherein the method continuously administered the formulation to achieve the therapeutic target of the anticoagulant from a standard of care treatment, or wherein the formulation at a dose rate such that the daily dose of an anticoagulants compound is equivalent to the bioavailable daily oral dose of a standard of care treatment, wherein the standard care of treatment is a bioavailable oral dose of 0.1 mg to 50 mg of the anticoagulants daily.

3. The method of claim 1 wherein the method continuously delivered the formulation to achieve a blood level of the anticoagulant by avoiding the GI tract that is equivalent to the blood level at a time point from 4 hr to 24 hrs obtained from once a daily bioavailable oral dose of 0.1 to 50 mg of the anticoagulant agents.

4. The method of claim 1 wherein the method continuously administers the formulation to achieve a blood level of the anticoagulant compound that is equivalent to the blood level at 12 hours obtained from once daily bioavailable oral dose of 0.1-50 mg of the anticoagulant agent.

5. The method of claim 1 wherein continuous administration of the formulation comprising the anticoagulant compound and the pharmaceutically acceptable carrier comprises continuous administration of the formulation for one day, two days, three days, four days, five days, six days, seven days, eight days, nine days, ten days, eleven days, twelve days, thirteen days, fourteen days, two weeks, three weeks, a month, or more than a month.

6. The method of claim 1 wherein the anticoagulant compound is selected from the group consisting of either factor Xa inhibitor such as Apixaban, Rivaroxaban, edoxaban, betrixaban and/or thrombin inhibitor such as dabigatran.

7. The method of claim 1 wherein the anticoagulant agent is apixaban.

8. The method of claim 1 wherein the continuous administration comprises continuous administration of apixaban to the patient at a rate of about 500-10000 μg/24 hr.

9. The method of claim 1 wherein continuous administration comprises continuous administration of the formulation to the subject to achieve a steady state plasma level of apixaban in a range of about 3-500 μg/L.

10. The method of claim 1 wherein apixaban is continuously delivered at a rate of 500 μg to 10000 μg/24 hour.

11. The method of claim 1 wherein the method achieves a steady state blood level of apixaban in the range of about 1-250 μg/L.

12. The method of claim 1 wherein apixaban is continuously delivered at a rate of 0.5 μg to 300 μg/hour.

13. The method of claim 1 wherein the method achieves a steady state blood level of apixaban in the range of about 3-150 μg/L.

14. The method of claim 1 wherein apixaban is continuously delivered at a rate of 100 g to 400 μg/hour for treating thromboembolic conditions.

15. The method of claim 1 wherein apixaban is continuously delivered at a rate of 500 g to 50000 μg/24 hour for preventing the risk of stroke and embolism in subject with atrial fibrillation.

16. The method of claim 1 wherein apixaban is continuously delivered at a rate of 500 g to 50000 μg/24 hour for preventing deep vein thrombosis.

17. The method of claim 1 wherein apixaban is continuously delivered at a rate of 500 g to 50000 μg/24 hour for preventing pulmonary embolism.

18. The method of claim 1 wherein apixaban is continuously delivered at a rate of 500 g to 50000 μg/24 hour for treating deep vein thrombosis.

19. The method of claim 1 wherein apixaban is continuously delivered at a rate of 500 g to 50000 μg/24 hour for treating pulmonary embolism.

20. The method of claim 1 wherein apixaban is continuously delivered at a rate of 500 g to 50000 μg/24 hour for reducing the risk of recurring deep vein thrombosis.

21. The method of claim 1 wherein apixaban is continuously delivered at a rate of 500 g to 50000 μg/24 hour for reducing the risk of recurring pulmonary embolism.

22. The method of claim 1 wherein apixaban is continuously delivered at a rate of 500 g to 50000 μg/24 hour for preventing GI tract bleeding.

23. The method of claim 1 wherein apixaban is continuously delivered at a rate of 500 g to 50000 μg/24 hour for reducing the GI tract bleeding.

24. The method of claim 1 wherein the method achieves a steady state blood level of apixaban in the range of about 1-400 μg/L.

25. The method of claim 1 wherein the pharmaceutically subcutaneous formulation of apixaban having at least one acceptable carrier comprising water, n-methyl-2-pyrrolidone, polyvinylpyrrolidone, carboxymethyl cellulose (CMC), Tween 80, dimethyl sulfoxide (DMSO), ethanol, 2-hydroxypropyl-β-cyclodextrin, dextrose, PEG400, citric acid, sodium bicarbonate, and/or combinations thereof.

26. The method of claim 1 wherein the pharmaceutically acceptable carrier comprises water, n-methyl-2-pyrrolidone, polyvinylpyrrolidone, citric acid, and/or sodium bicarbonate.

27. The method of claim 1 wherein the subcutaneous delivery system, wherein the subcutaneous delivery system comprises a pump for subcutaneous infusion of the DOACs compound via an external drug supply.

28. The method of claim 1 comprising a continuous administration of apixaban through the transdermal formulation to the subject.

29. The method of claim 1 wherein a transdermal delivery system of DOACs compound, comprises the DOACs compound and carrier, wherein the DOACs compound comprises apixaban, rivaroxaban, edoxaban, betrixaban, dabigatran and/or combination thereof.

30. A transdermal delivery system for preventing and/or treating thromboembolic conditions by continuous administration of the treatment of the formulations comprising a Direct Oral Anticoagulants (DOACs) compound and a pharmaceutically acceptable carrier, where the DOACs compound is selected from the group consisting of DOACs, and wherein the transdermal delivery system continuously administers the formulation to the subject via transdermal and/or subcutaneous and/or intramuscular and/or buccal route and/or by avoiding the gastrointestinal (GI) tract exposure.

31. The transdermal delivery system of claim 30, wherein the transdermal delivery system continuously administers the formulation to achieve the therapeutic target of the anticoagulant from a standard of care treatment, or wherein the formulation at a dose rate such that the daily dose of an anticoagulants compound is equivalent to the bioavailable daily oral dose of a standard of care treatment, wherein the standard care of treatment is a bioavailable oral dose of 0.1 mg to 50 mg of the anticoagulants daily.

32. The transdermal delivery system of claim 30 wherein the transdermal delivery system continuously delivers the formulation to achieve a blood level of the anticoagulant by avoiding the GI tract that is equivalent to the blood level at a time point from 4 hr to 24 hrs obtained from once a daily bioavailable oral dose of 0.1 to 50 mg of the anticoagulant agents.

33. The transdermal delivery system of claim 30 wherein the transdermal delivery system continuously administers the formulation to achieve a blood level of the anticoagulant compound that is equivalent to the blood level at 12 hours obtained from once daily bioavailable oral dose of 0.1-50 mg of the anticoagulant agent.

34. The transdermal delivery system of claim 30 wherein continuous administration of the formulation comprising the anticoagulant compound and the pharmaceutically acceptable carrier comprises continuous administration of the formulation for one day, two days, three days, four days, five days, six days, seven days, eight days, nine days, ten days, eleven days, twelve days, thirteen days, fourteen days, two weeks, three weeks, a month, or more than a month.

35. The transdermal delivery system of claim 30 wherein the anticoagulant compound is selected from the group consisting of either factor Xa inhibitor such as Apixaban, Rivaroxaban, edoxaban, betrixaban and/or thrombin inhibitor such as dabigatran.

36. The transdermal delivery system of claim 30 wherein the anticoagulant agent is apixaban.

37. The transdermal delivery system of claim 30 wherein the continuous administration comprises continuous administration of apixaban to the patient at a rate of about 500-10000 μg/24 hr.

38. The transdermal delivery system of claim 30 wherein continuous administration comprises continuous administration of the formulation to the subject to achieve a steady state plasma level of apixaban in a range of about 3-500 μg/L.

39. The transdermal delivery system of claim 30 wherein apixaban is continuously delivered at a rate of 500 μg to 10000 μg/24 hour.

40. The transdermal delivery system of claim 30 wherein the transdermal delivery system achieves a steady state blood level of apixaban in the range of about 1-250 μg/L.

41. The transdermal delivery system of claim 30 wherein apixaban is continuously delivered at a rate of 0.5 μg to 300 μg/hour.

42. The transdermal delivery system of claim 30 wherein the transdermal delivery system achieves a steady state blood level of apixaban in the range of about 3-150 μg/L.

43. The transdermal delivery system of claim 30 wherein apixaban is continuously delivered at a rate of 100 μg to 400 μg/hour for treating thromboembolic conditions.

44. The transdermal delivery system of claim 30 wherein apixaban is continuously delivered at a rate of 500 pg to 50000 μg/24 hour for preventing the risk of stroke and embolism in subject with atrial fibrillation.

45. The transdermal delivery system of claim 30 wherein apixaban is continuously delivered at a rate of 500 pg to 50000 μg/24 hour for preventing deep vein thrombosis.

46. The transdermal delivery system of claim 30 wherein apixaban is continuously delivered at a rate of 500 μg to 50000 μg/24 hour for preventing pulmonary embolism.

47. The transdermal delivery system of claim 30 wherein apixaban is continuously delivered at a rate of 500 μg to 50000 μg/24 hour for treating deep vein thrombosis.

48. The transdermal delivery system of claim 30 wherein apixaban is continuously delivered at a rate of 500 pg to 50000 μg/24 hour for treating pulmonary embolism.

49. The transdermal delivery system of claim 30 wherein apixaban is continuously delivered at a rate of 500 μg to 50000 μg/24 hour for reducing the risk of recurring deep vein thrombosis.

50. The transdermal delivery system of claim 30 wherein apixaban is continuously delivered at a rate of 500 μg to 50000 μg/24 hour for reducing the risk of recurring pulmonary embolism.

51. The transdermal delivery system of claim 30 wherein apixaban is continuously delivered at a rate of 500 μg to 50000 μg/24 hour for preventing GI tract bleeding.

52. The transdermal delivery system of claim 30 wherein apixaban is continuously delivered at a rate of 500 μg to 50000 μg/24 hour for reducing the GI tract bleeding.

53. The transdermal delivery system of claim 30 wherein the transdermal delivery system achieves a steady state blood level of apixaban in the range of about 1-400 μg/L.

54. The transdermal delivery system of claim 30 wherein the pharmaceutically subcutaneous formulation of apixaban having acceptable carrier comprises of water, n-methyl-2-pyrrolidone, polyvinylpyrrolidone, carboxymethyl cellulose (CMC), Tween 80, dimethyl sulfoxide (DMSO), ethanol, 2-hydroxypropyl-p-cyclodextrin, dextrose, PEG400, citric acid, sodium bicarbonate, and/or combinations thereof.

55. The transdermal delivery system of claim 30 wherein the pharmaceutically acceptable carrier comprises water n-methyl-2-pyrrolidone, polyvinylpyrrolidone, citric acid, and/or sodium bicarbonate.

56. The transdermal delivery system of claim 30 wherein the subcutaneous delivery system, wherein the subcutaneous delivery system comprises a pump for subcutaneous infusion of the DOACs compound via an external drug supply.

57. The transdermal delivery system of claim 30 wherein a continuous administration of apixaban through the transdermal formulation to the subject

58. The transdermal delivery system of claim 30 wherein a transdermal delivery system of DOACs compound, comprises the DOACs compound and carrier, wherein the DOACs compound comprises apixaban, rivaroxaban, edoxaban, betrixaban, dabigatran and/or combination thereof.

59. The transdermal delivery system of claim 30 wherein the transdermal delivery system is an occlusive or non-occlusive transdermal drug delivery formulation, wherein the occlusive and/or non-occlusive transdermal drug delivery formulation comprises a liquid, a semisolid, a dispersion, a suspension, an oil-in-water emulsion, a water-in-oil emulsion, a polymer film, a patch, a drug-in-adhesive, a matrix, a metered dose transdermal spray for topical application, a metered dose transdermal formulations for topical application, or a combination thereof

60. The transdermal delivery system of claim 30 wherein the transdermal delivery system further comprises microneedles.

61. The transdermal delivery system of claim 30 wherein the transdermal delivery system is a transdermal patch.

62. The transdermal delivery system of claim 30 wherein the transdermal patch comprises a reservoir patch, a microreservoir patch, a matrix patch, a drug-in-adhesive patch, a pressure sensitive adhesive patch, an extended-release transdermal film, a multilayer matrix patch, a multilayer reservoir patch, a transdermal patch with an overlay adhesive, and/or combinations thereof.

63. The transdermal delivery system of claim 30 wherein the delivery of the DOACs compound is continuous over the treatment cycle.

64. The transdermal delivery system of claim 30 wherein the transdermal delivery system continuously administers the DOACs compound to achieve an AUC of the DOACs compound of between 80% and 120% of the exposure (AUC) obtained from a standard of care treatment by avoiding peak and valley in plasma concentration, or wherein the transdermal delivery system continuously administers the DOACs compound at a dose rate such that the daily dose of the DOACs compound is equivalent to the bioavailability of the daily oral dose of a standard of care treatment, wherein the standard of care treatment is an oral dose of 1 mg to 250 mg of the DOACs compound once daily.

65. The transdermal delivery system of claim 30 wherein the transdermal delivery system continuously administers the DOACs compound to achieve a blood level of the DOACs compound that is equivalent to the blood level at a time point from 5 hours to 24 hours obtained from once daily oral dose of 1-500 mg of the DOACs compound.

66. The transdermal delivery system of claim 30 wherein the transdermal delivery system continuously administers the DOACs compound to achieve a blood level of the DOACs compound that is equivalent to the blood level at 12 hours obtained from once daily oral dose of 1 mg to 500 mg of the DOACs compound.

67. The transdermal delivery system of claim 30 wherein the transdermal delivery system continuously administers the DOACs compound to a subject for one day, two days, three days, four days, five days, six days, seven days, eight days, nine days, ten days, eleven days, twelve days, thirteen days, or fourteen, or twenty-eight days.

68. The transdermal delivery system of claim 30 wherein the transdermal delivery system delivers the DOACs compound to a subject at a rate of 10 μg/hour to 2000 μg/hour.

69. The transdermal delivery system of claim 30 wherein the transdermal delivery system delivers the DOACs compound to a subject to achieve a steady state plasma level of apixaban in a range of 1 μg/L to 500 μg/L.

70. The transdermal delivery system of claim 30 wherein the DOACs compound is continuously delivered at a rate of 15 μg/hour to 2500 μg/hour.

71. The transdermal delivery system of claim 30 wherein a steady state blood level of the DOACs compound in the range of 1 μg/L to 250 μg/L is achieved.

72. The transdermal delivery system of claim 30 wherein the DOACs compound is continuously delivered at a rate of 15 μg/hour to 1500 μg/hour.

73. The transdermal delivery system of claim 30 wherein a steady state blood level of the DOACs compound in the range of 1 μg/L to 100 μg/L is achieved.

74. The transdermal delivery system of claim 30 wherein the DOACs compound is continuously delivered at a rate of 15 μg/hour to 500 μg/hour.

75. The transdermal delivery system of claim 30 wherein a steady state blood level of the DOACs compound in the range of 1 μg/L to 50 μg/L is achieved.

76. The transdermal delivery system of claim 30 wherein the carrier comprises a polymer, an adhesive, a plasticizer, a solvent, a solubilizer, a diluent, a suspending agent, a dispersing agent, a crystallization inhibitor, a gelling agent, a penetration enhancer, a pH adjusting agent, a buffering agent, a pH stabilizer, an emulsifying agent, a surfactant, a suspending agent, a stabilizer, a preservative, a chelating agent, a complexing agent, an emollient, a humectant, a demulcent, a skin irritation reducing agent, an antioxidant, an oxidant, a tackifier, a filler, or a combination thereof.

77. The transdermal delivery system of claim 30 wherein the matrix is selected from the group consisting of natural polymers, polysaccharides. agar, alginic acid and derivatives, cassia tora, collagen, gelatin, gellum gum, guar gum, pectin, potassium cargeenan, sodium carageenan, tragacanth, xantham, gum copal, chitosan, resin, semisynthetic polymers, cellulose, methylcellulose, ethyl cellulose, carboxymethyl cellulose, hydroxylpropyl cellulose, hydroxylpropylmethyl cellulose, synthetic polymers, carboxyvinyl polymers, carbomers, carbopol 940, carbopol 934, carbopol 971p NF, polyethylene, clays, silicates, bentonite, silicon dioxide, polyvinyl alcohol, acrylic polymers (eudragit), acrylic acid esters, polyacrylate copolymers, polyacrylamide, polyvinyl pyrrolidone homopolymer, polyvinyl pyrrolidone copolymers, PVP, Kollidon 30, poloxamer, isobutylene, ethyl vinyl acetate copolymers, natural rubber, synthetic rubber, pressure sensitive adhesives, silicone polymers, bio psa 4302, bio-psa 4202, acrylic pressure sensitive adhesives, duro-tak 87-2156, duro-tak 387-2287, duro-tak 87-9301, duro-tak 387-2051, polyisobutylene, polyisobutylene low molecular weight, polyisobutylene medium molecular weight, polyisobutylene 35000 mw, acrylic copolymers, rubber based adhesives, hot melt adhesives, styrene-butadiene copolymers, bentonite, all water and/or organic solvent swellable polymers and combinations thereof.

78. The transdermal delivery system of claim 30 wherein the apixaban is present in a concentration in the range of from 0.1-50 wt %, preferably from 1-30 wt %, more preferably 1-20 wt %, in each case relative total mass of the active substance reservoir.

79. The transdermal delivery system of claim 30 wherein apixaban is present in the active substance reservoir either in dissolved and/or suspended and/or dispersed and/or combinations thereof.

80. The transdermal delivery system of claim 30 wherein the active substance reservoir contains at least one solubilizer, preferably in an amount of from 1 to 99 wt %, with particular preference from 5 to 70 wt %, in each case relative to the total weight of the active substance reservoir.

81. The transdermal delivery system of claim 30 wherein the solubilizer is selected from the group consisting of methanol, ethanol, isopropyl alcohol, butanol, propanol, polyhydric alcohols, glycols, propylene glycol, polyethylene glycol, dipropylene glycol, hexylene glycol, butyene glycol, glycerine, derivative of glycols, pyrrolidone, N methyl 2-pyrrolidone, 2 pyrrolidone, sulfoxides, dimethyl sulfoxide, decymethylsulfoxide, dimethylisosorbide, mineral oils, vegetable oils, sesame oil water, polar solvents, semi polar solvents, non polar solvents, volatile chemicals, ethanol, propanol, ethyl acetate, acetone, methanol, dichloromethane, chloroform, toluene, IPA, hexane, acids, acetic acid, lactic acid, levulinic acid, bases, pentane, dimethylformamide, butane, lipids, and combinations thereof.

82. The transdermal delivery system of claim 30 wherein the active substance reservoir contains at least one permeation-enhancing agent, in an amount of from 0.1 to 50 wt %, with particular reference from 1 to 25 wt %, in each case relative to the total weight of the active substance reservoir.

83. The transdermal delivery system of claim 30 where in the permeation-enhancing agent is selected and is selected from the group consisting of dimethylsulfoxide, dimethylacetamide, dimethylformamide, decymethylsulfoxide, dimethylisosorbide, azone, pyrrolidones, N-methyl-2-pyrrolidone, 2-pyrrolidon, esters, fatty acid esters, propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate, fatty acids, capric acid, caprylic acid, lauric acid, oleic acid, myristic acid, linoleic acid, stearic acid, palmitic acid, alcohols, fatty alcohols, glycols, oleyl alcohol, nathanol, dodecanol, propylene glycol, glycerol, ethers, alcohol, diethylene glycol monoethyl ether, urea, triglycerides, triacetin, polyoxyethylene fatty alcohol ethers, polyoxyethylene fatty acid esters, esters of fatty alcohols, essential oils, surfactant type enhancers, brij, sodium lauryl sulfate, tween, polysorbate, terpene, terpenoids, and combinations thereof.

84. The transdermal delivery system of claim 30 wherein a pH of the composition ranges from about 3.0 to about 9.0 prior to administration.

85. The transdermal delivery system of claim 30 wherein the DOACs compound comprises apixaban, rivaroxaban, or a combination thereof, the polar aprotic solvent comprises n-methyl-2-pyrrolidone, 2-pyrrolidone, dimethyl sulfoxide, dimethylformamide, dimethylacetamide, acetone, acetonitrile, tetrahydrofuran, or a combination thereof, and the soluble polymer is water soluble and/or water insoluble comprises thereof.

86. The transdermal delivery system of claim 30 wherein the composition has an osmolality ranging from about 250 mOsm/kg to about 1600 mOsm/kg.

87. The transdermal delivery system of claim 30 wherein the parenteral administration is intramuscular, intravenous, subcutaneous, depot, intraarterial, intraperitoneal, infusion, or by implant administration.

88. The transdermal delivery system of claim 30 wherein the parenteral administration is a subcutaneous infusion, further wherein the subcutaneous infusion is continuous, pulsatile, or intermittent with an uninterrupted drug supply from an external drug supply, wherein the external drug supply is not disconnected during the parenteral administration except when necessary to change or replenish the formulation or when treatment is completed as determined by a medical professional.

89. The transdermal delivery system of claim 30 further comprising an excipient, wherein the excipient comprises a solvent, a solubilizer, a diluent, a suspending agent, a dispersing agent, gelling agent, polymer, penetration enhancer, plasticizer, pH adjusting agent, pH stabilizer, emulsifying agent, a cyclodextrin and derivatives thereof, a surfactant, a preservative, a chelating agent, a complexing agent, an emollient, a humectant, a demulcent, a skin irritation reducing agent, tonicity agent, buffers, an antioxidant, an oxidant, a tackifier, a filler, a crystallization inhibitor, a volatile chemical, or a combination thereof.

90. The method of claim 1, comprising a continuous administration of apixaban through the subcutaneous and/or intramuscular formulation to the subject

91. The method of claim 90, wherein the continuous administration composition comprising an DOACs compound, a polar aprotic solvent, and a soluble polymer.
